(19) United States
(12) Patent Application Publication
(10) Pub. No.: US 2016/0120643 A1
(43) Pub. Date: May 5, 2016

(54) TRANSCATHETER CARDIAC VALVE PROSTHETIC

(71) Applicant: Tara Kupumbati, Irvine, CA (US)

(72) Inventor: Tara Kupumbati, Irvine, CA (US)

(21) Appl. No.: 14/689,833

(22) Filed: Apr. 17, 2015

Related U.S. Application Data
(60) Provisional application No. 62/075,727, filed on Nov. 5, 2014.
(60) Provisional application No. 62/075,734, filed on Nov. 5, 2014.

(51) Int. Cl. A61F 2/24; A61F 2/966

(52) U.S. Cl. CPC A61F 2/2418; A61F 2/2412; A61F 2/243; A61F 2/2436; A61F 2/966

(57) ABSTRACT

A cardiac valve prosthetic effective for use in a living mammalian heart without inducing atrial fibrillation. A plurality of valve leaflets is internally disposed in a radially, compressible stent frame. A tubular valve portion is connected to a tubular inferior skirt portion to form the stent frame. Optionally, a superior portion is fluidly connected to the valve portion. An asymmetrical bottom edge of the inferior skirt portion is formed by an upper edge section interconnected to a lower edge section. A first distance between the upper edge section and a top edge of the valve portion is shorter that a second distance between the lower edge section and the top edge of the valve portion.

### CROSS REFERENCE

This application claims priority to U.S. Provisional Patent Application No. 62/075,727 filed on Nov. 5, 2014, and to U.S. Provisional Patent Application No. 62/075,734 filed on Nov. 5, 2014 the specification(s) of which is/are incorporated herein in their entirety by reference.

### FIELD OF THE INVENTION

The present invention relates to medical devices for a heart, in particular, a cardiac valve prosthetic for transcatheter delivery. The cardiac valve prosthetic may be used to replace native heart valves to treat valve insufficiency, such as treating mitral valve insufficiency or mitral regurgitation. The cardiac valve prosthetic is effective for preventing atrial fibrillations after mounting the prosthetic at the site of a damaged native valve.

### BACKGROUND OF THE INVENTION

The human heart has four chambers: right atrium, right ventricle, left atrium, and left ventricle. The right atrium receives blood from the veins and pumps it to the right ventricle. The right ventricle receives the blood from the right atrium and pumps it to the lungs for oxygenation. The left atrium receives oxygenated blood from the lungs and pumps it to the left ventricle. The left ventricle pumps oxygenated blood to the rest of the body.

There are four heart valves which play important roles in moving oxygenated and de-oxygenated blood within and out of the heart: the tricuspid, bicuspid (mitral), pulmonary, and aortic valves. All these valves acts as one-way valves, allowing blood to flow either from one chamber to another or allowing blood to flow out of the heart, in only one direction. The valves control the flow of blood through the heart by opening and closing during the contractions for the heart. The opening and closing functions of the valves are controlled by pressure differences generated within the heart, as well as some muscles located within the heart. The tricuspid valve is located between the right atrium and the right ventricle and it allows the flow of blood from right atrium to right ventricle. The bicuspid (mitral) valve is located between the left atrium and the left ventricle and it allows the flow of blood from the left atrium to the left ventricle. The pulmonary valve is located at the base of the pulmonary artery and allows the flow of blood from the right ventricle to the pulmonary artery. The aortic valve is located at the base of the aorta and allows the blood flow from the left ventricle to the aorta.

The cardiac skeleton (fibrous skeleton of the heart) is a high density single structure of connective tissue that forms and anchors the four valves and influences the forces exerted through them. The cardiac skeleton separates and partitions the atria from the ventricles, thereby forming the primary channel that electrical energy follows from the top to the bottom of the heart. The deformation of valvular annulus or cardiac skeleton can potentially influence the electric energy flow in the heart. Therefore, It is important to maintain the innate structure of this cardiac skeleton and the annulus of the valves.

Valve stenosis, or stenosis, is a common heart condition where a heart valve is unable to open properly due to thickening, stiffening or fusing of the valve leaflets. This condition may be caused by aging or by valve malformation. The four types of stenosis are aortic stenosis, mitral stenosis, pulmonary stenosis, and tricuspid stenosis. Aortic stenosis is the most common form and is usually caused by calcium buildup or scarring that develop during aging. Leaking of a heart valve is another common heart problem known as regurgitation.

One method of treating stenosis, or regurgitation, is by replacing the damaged valve with an artificial valve by means of open-heart surgery. Since complications may develop after open-heart surgery, alternative methods, such as minimally invasive surgery or a catheter procedure, are preferred. For example, transcatheter aortic valve implantation (TAVI) is a minimally invasive surgical procedure where an artificial valve is inserted at the site of the native valve via a catheter, without having to remove the native valve. The artificial valve is usually collapsed as it is delivered to the native valve site, and then expanded into place.

One drawback of TAVI is the development of new-onset atrial fibrillations (NOAF) in some patients. Atrial fibrillation (AF) is described as an irregular heart rate caused by the atria chamber beating chaotically and out of sync with the ventricle, thereby resulting in poor blood flow in a body. ¹A study by Amat-Santos et al., reports that of the 138 patients who underwent TAVI using the Edwards SAPIEN prosthesis (Edwards Lifesciences, Irvine, Calif.). NOAF occurred in about one-third of the patients having no prior history of AF.

Current artificial valves, such as the Medtronic CoreValve prosthesis (Medtronic, Minneapolis, Minn.) and the aforementioned Edwards SAPIENS prosthetic, may cause AF after valve replacement. Hence, there is a need for a modified artificial valve that does not induce AF.

For example, one aspect of the present invention is the use of the modified artificial valve to supplement the mitral valve. However, the heart valve prosthesis design disclosed herein is not limited to the mitral valve. The present invention may also be used to treat other heart valve insufficiencies such as tricuspid, pulmonary, and aortic valves. It may also be used to treat any other valves having biological fluid movements that are regulated via valves.

The mitral valve is composed of two leaflets (the anterior and posterior), which are located at the mitral annulus, the annulus being the ring that forms the junction between the left atrium and the left ventricle. The mitral leaflets are tethered to papillary muscles of the left ventricle via chordae tendineae. The chordae tendineae prevents the mitral leaflets from averting into the left atrium during systole.

Mitral valve disease is one of the common heart valve diseases caused by various reasons. The common symptom of the disease is mitral valve regurgitation (mitral valve insufficiency). Although there are different reasons for mitral valve insufficiency, such as mitral annulus dilation, left ventricle dilation, mitral leaflet prolapse, degeneration of papillary muscles and/or chordae tendineae, the outcome is mitral valve regurgitation. It is a condition in which the mitral valve does not close completely, resulting in the backflow of blood from the left ventricle to the left atrium

Surgical methods and a few transcatheter devices are currently available to treat mitral regurgitation The surgical methods involve open-heart surgery to repair prolapsed leaflets and/or implantation of various prosthetic devices such as annuloplasty rings/bands, repairing devices, and even prosthetic heart valves. The transcatheter methods/devices may involve the delivery of clips to repair leaflets, placing cinch devices in the leaflets, reshaping the mitral annulus via devices implanted in the coronary sinus, or delivery of radiofrequency to ablate the leaflets to name a few. Although some of these transcatheter methods are in use currently, they have their limitations. Recent developments in an effort to repair or replace mitral valve include less invasive transcatheter techniques to deliver a fully functional replacement prosthetic mitral valve. Here, the prosthetic mitral valve is mounted on the tip of the delivery system in a compressed state and advanced through a blood vessel or the body of the patient to the implant site. At the implant site, the prosthetic valve is positioned and expanded to act as a functional mitral valve.

While these devices may replace the mitral valve, some of the challenges that are associated with these devices include the lack of preservation of the native structures surrounding the mitral valve and the cardiac skeleton as well. These devices are designed to have sufficient radial force to push away the native mitral leaflets and stay in position. The deployed devices can impact neighboring structures of the mitral annulus that is the cardiac skeleton and may interfere with the electrical system of the heart. More importantly, these devices may obstruct the aortic valve from the left ventricle side.

The present invention will address the above mentioned challenges as well as preserve the native structure of the mitral valve, thereby preserving the cardiac skeleton and neighboring aorta.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BACKGROUND ART

Replacement of bioprosthetic heart valves via catheter and without open-heart surgery has been developed and in use for over a decade for aortic heart valves (TAVI). These bioprosthetic valves are delivered successfully via transfemoral approach as well as transapical approaches. Recent developments are focused on developing bioprosthetic valves to be used as replacement heart valves for mitral valve insufficiency and to be implanted without open-heart surgery. The relevant prior arts to this invention are described below.

U.S. 2012/0035722 of Tuval describes bioprosthetic heart valves to be implanted (via transapically, transatrially, and trans-septally) in the mitral position. The prosthesis includes a self-expanding frame and two or more support arms. The valve prosthesis is sutured to the self-expanding frame. Each support arm corresponds to the native leaflets and at least one support arm immobilizes the native leaflets and holds the native leaflet close to the main frame.

U.S. 2009/0276040 of Rowe describes a prosthetic mitral valve assembly and method of inserting the same. The prosthetic mitral valve has a flared upper end and tapered portion that can fit into the native mitral valve. It can include a stent or outer support frame with a valve mounted therein. It can be expanded radially outwardly and into contact with the native tissue to create a pressure fit. The prosthesis is deployed in couple of positions: 1. Below the annulus of the mitral valve so that the annulus prevents the upward movement of the prosthesis into left atrium; and 2. Positioned in such a way that leaflet of the native mitral valve hold the assembly and prevent downward movement of the prosthesis.

U.S. 2012/0101572 of Kovalsky describes the mitral bioprosthesis with low ventricular profile and a stem-like support structure. The support structure includes an upstream section that is outwardly expandable to sit against the atrial wall so as to at least partially anchor the prosthesis there and a downstream section to which the prosthetic mitral valve is coupled that extends between the left atria and the ventricle through the native mitral valve.

U.S. Pat. No. 8,449,599 of Chau describes a prosthetic valve for replacing mitral valve, consisting of a radially compressible main body, one way valve portion, and at least one ventricular anchor attached to main body. The space between the main body and the frame is designed to receive the native mitral leaflet. The prosthetic valve also includes atrial sealing member adapted for placement above the annulus of the native mitral valve

Lastly, U.S.Pat. No. 8,579,964 of Lane describes a transcatheter mitral prosthesis comprising an anchor, an atriai skirt, an annular region, and a ventricular skirt. The prosthetic valve also has a plurality of valve leaflets, each having a first end and free end. The first end of the leaflet is coupled to the anchor. The free end of the leaflets may open and close to perform the function of the native mitral leaflet.

### SUMMARY OF THE INVENTION

The present invention features a cardiac valve prosthetic effective for use in a living mammalian heart without inducing atrial fibrillation. In one embodiment, the cardiac valve prosthetic comprises a radially, collapsible stent frame and a plurality of valve leaflets internally disposed in the stent frame. The prosthetic may further comprise a plurality of anchors. The stent frame comprises at least a valve portion and an inferior skirt portion, and may further comprise a superior portion.

In another embodiment, the present invention may also feature a cardiac valve prosthetic for implantation within a mitral valve annulus using a delivery system. The cardiac valve prosthetic may comprise a radially compressible stent frame, a plurality of valve leaflets, and a plurality of anchors disposed around the stent frame. The anchors may be directly or indirectly coupled to the stent frame. A superior portion of the compressible stent frame can seal the atrial side of the mitral valve when expanded. The inferior portion of the stent frame may preserve the innate structure of the mitral valve and the adjacent aortic valve. A section of the inferior portion nearest to the aortic valve side is eliminated.

### BRIEF DESCRIPTION OF THE DRAWINGS

FIG. 1 shows a perspective view of one embodiment of the present invention.

FIG. 2 shows a schematic of a cardiac skeleton showing four heart valves.

FIG. 3 shows a schematic of a heart with four chambers and four valves.

FIG. 4 shows a schematic of a heart with a normal mitral valve in a closed position.

FIG. 5 shows a schematic of degenerative mitral regurgitation caused by mitral valve prolapse. The mitral valve is not completely closed as compared to a normal mitral valve. The arrow shows the regurgitation during systole.

FIG. 6 shows a schematic of functional mitral regurgitation. The mitral leaflets, chordae, and papillary muscles appear normal, but the leaflets are not completely closed, thereby leading to regurgitation. The arrow shows the regurgitation during systole.

FIG. 7 shows a schematic of a heart with one embodiment of the present invention deployed in the mitral valve position.

FIG. 8 shows a longitudinal cross-section of one embodiment of the present invention deployed in a heart valve. The leaflets are in a closed position.

FIG. 9 shows a top view of an embodiment of the present invention.

FIG. 10 shows a side view of an embodiment of the present invention.

FIG. 11 shows a bottom view of an embodiment of the present invention.

FIG. 12 shows the different portions of one embodiment of the present invention.

FIG. 13 shows an exploded view of the different portions of one embodiment of the present invention.

FIGS. 14A-14D show side views of alternative embodiments of a superior portion of the present invention.

FIGS. 14E-14I show top views of alternative embodiments of the superior portion of the present invention.

FIGS. 15A-15D show side views of alternative embodiments of a medial valve portion of the present invention.

FIGS. 16A-16M show side views of alternative embodiments of an inferior skirt portion of the present invention.

FIGS. 17A-17F show side views of alternative embodiments of the present invention.

FIGS. 18A-18D show top views of alternative embodiments of the superior portion of the present invention.

FIGS. 19A-19D show bottom views of alternative embodiments of the inferior valve portion of the present invention.

FIG. 20 show a non-limiting example of anchors attached to the native valve annulus (shown is the mitral annulus).

FIGS. 21A-21K show non-limiting examples of anchors that may be used with the present invention.

FIG. 22 shows a non-limiting example of a delivery system. As known to one skilled in the art, there are various embodiments of a delivery system for delivering the transcatheter cardiac valve prosthesis or any other valve prosthesis. In some embodiments, the delivery system may comprise an advancing tip, a sheath that compresses the prosthesis, anchors, a sheath that compresses the anchors, an outer sheath, and a posterior portion.

### DESCRIPTION OF PREFERRED EMBODIMENTS

Following is a list of elements corresponding to a particular element referred to herein:
100 cardiac valve prosthetic
105 heart
106 cardiac skeleton
110 stent frame
120 valve portion or medial valve portion
121 top edge of a valve portion
130 inferior skirt portion
131 skirt sidewall
132 asymmetrical bottom edge
133 upper edge section
134 lower edge section
135 first distance
136 second distance
138 flap
140 valve leaflets
141 posterior end
142 distal end
150 superior portion
151 top edge of a superior portion
160 anchors
170 flexible material
200 catheter

As used herein, the use of the term “native” refers to the natural or biological, i.e. not artificial. As used herein, the terms “valve portion” and “medial valve portion” may be used interchangeably.

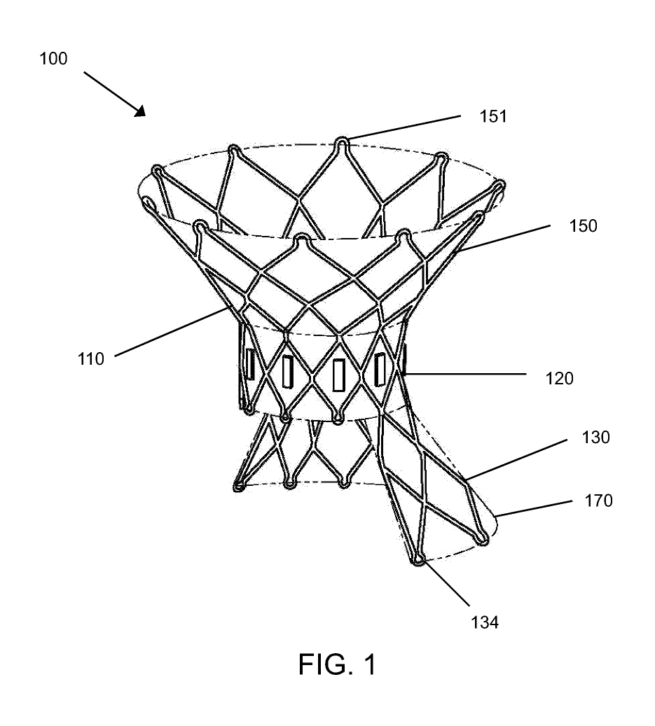

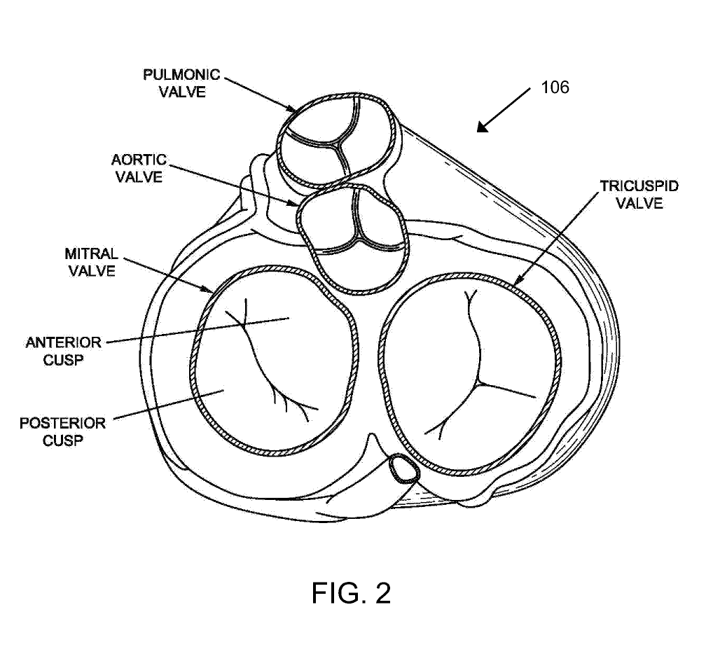

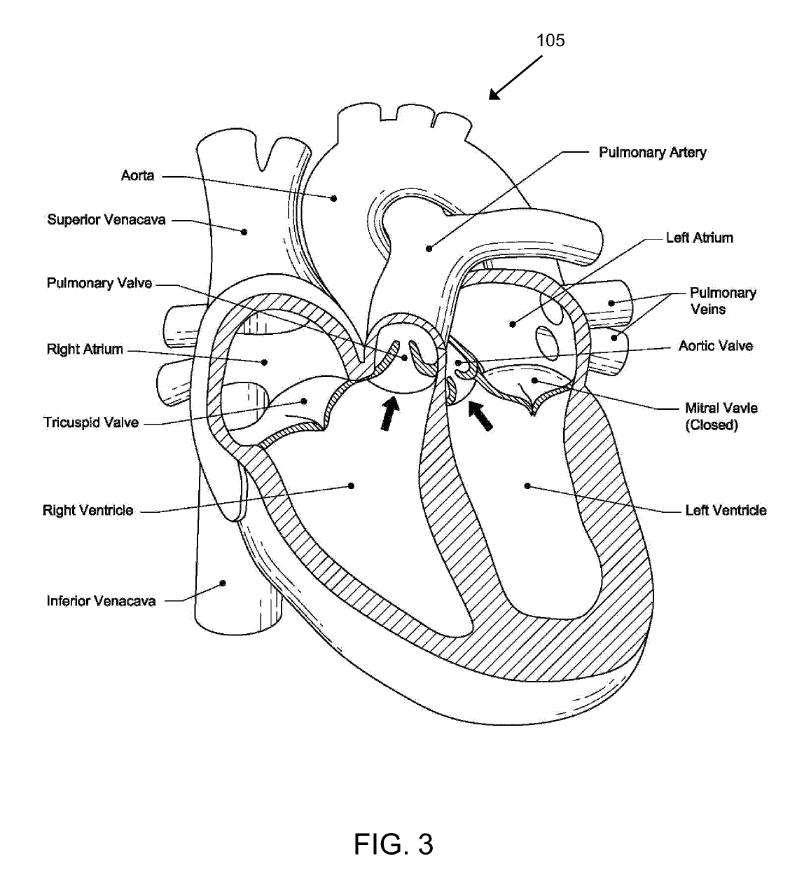

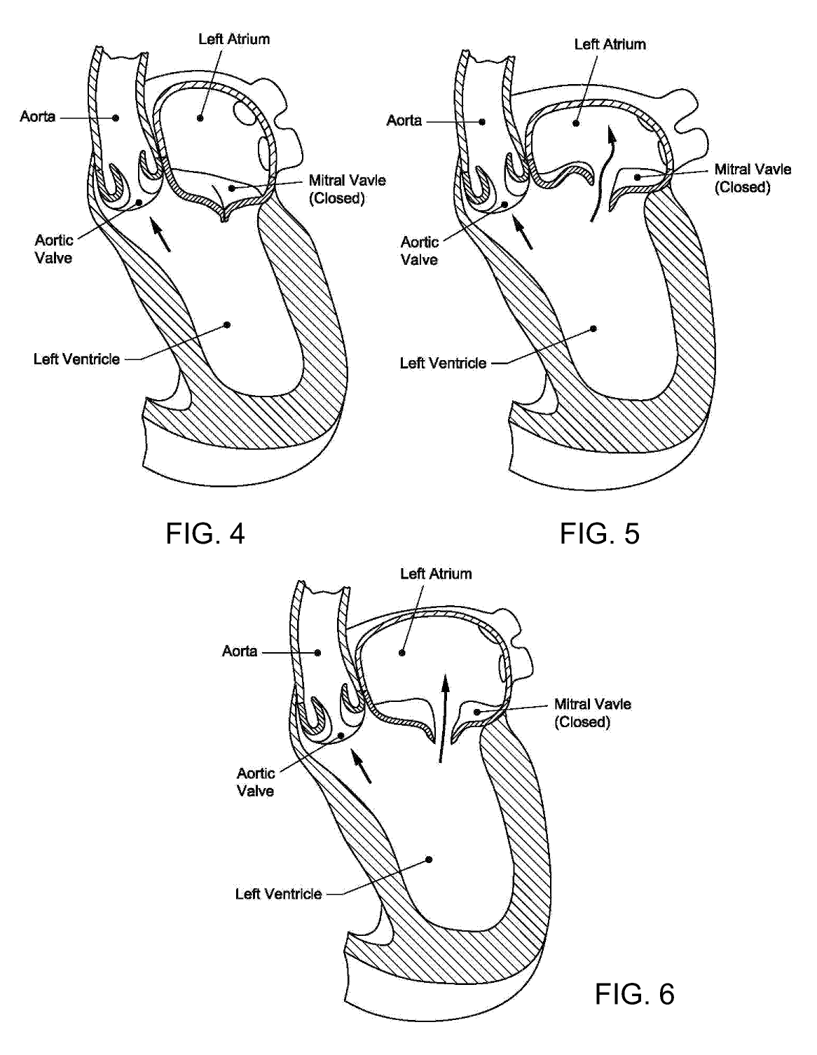

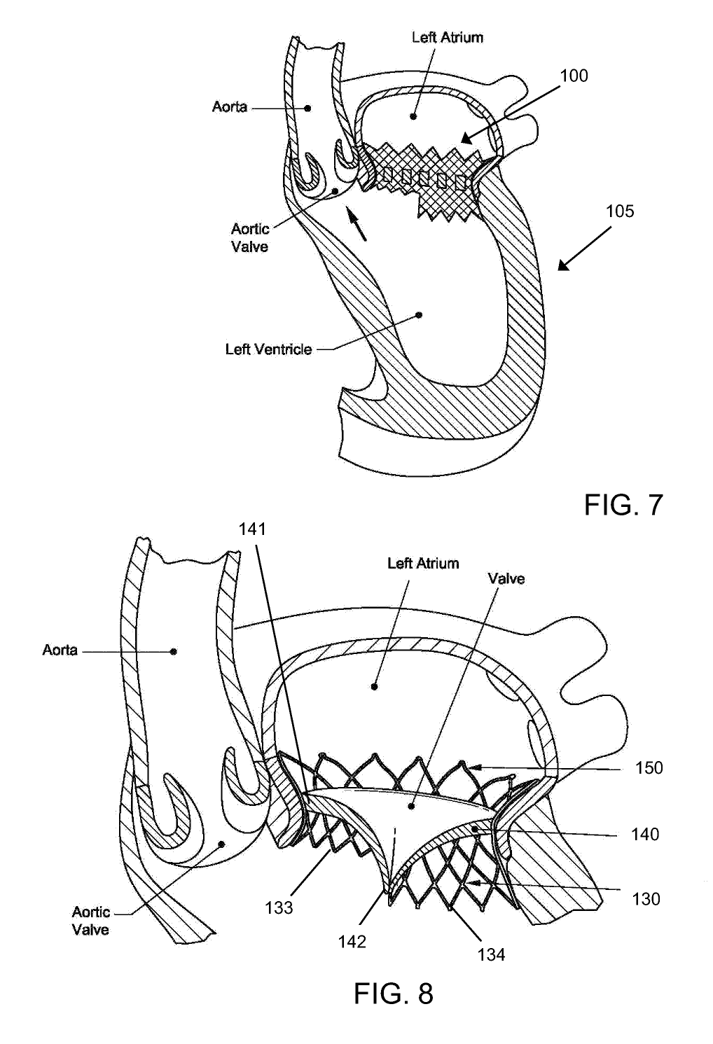

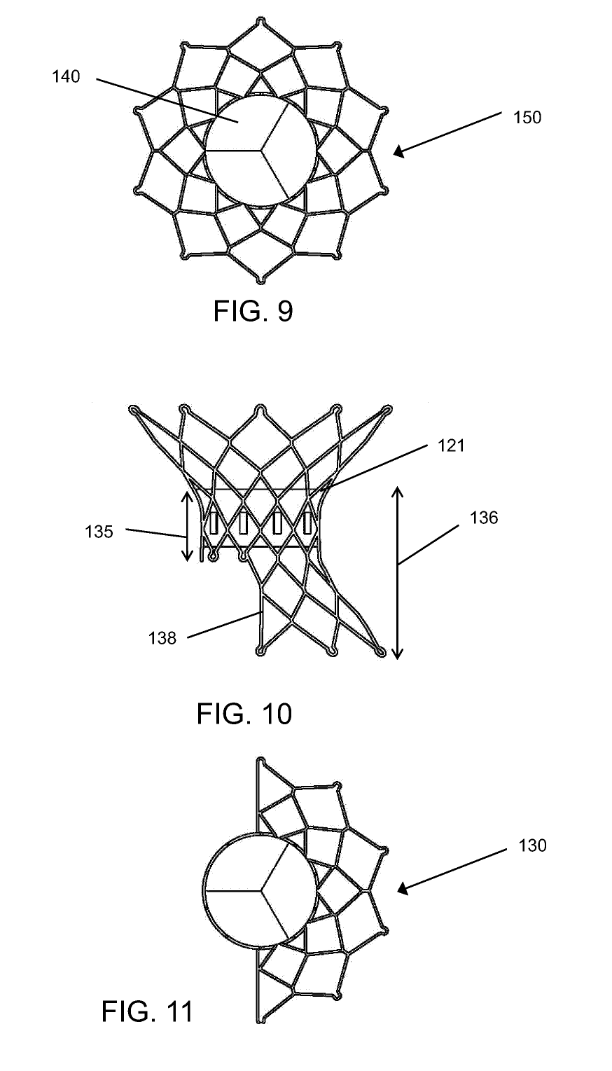

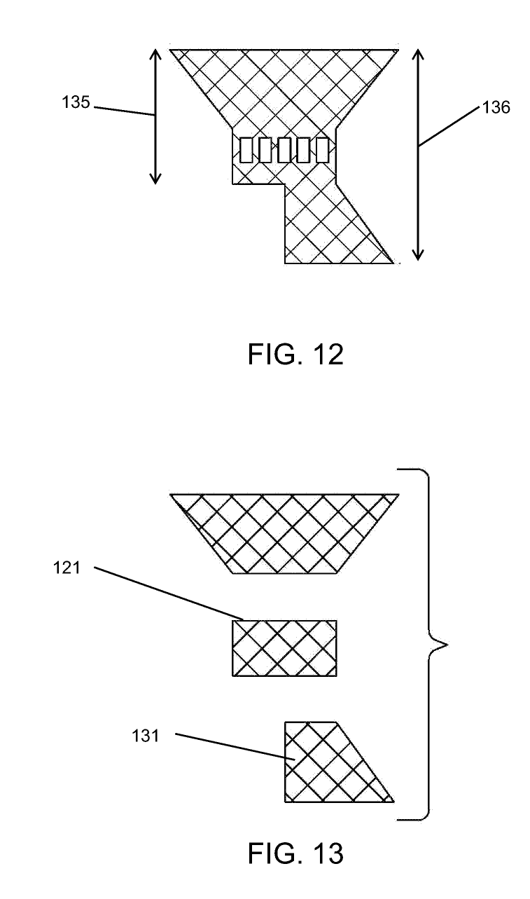

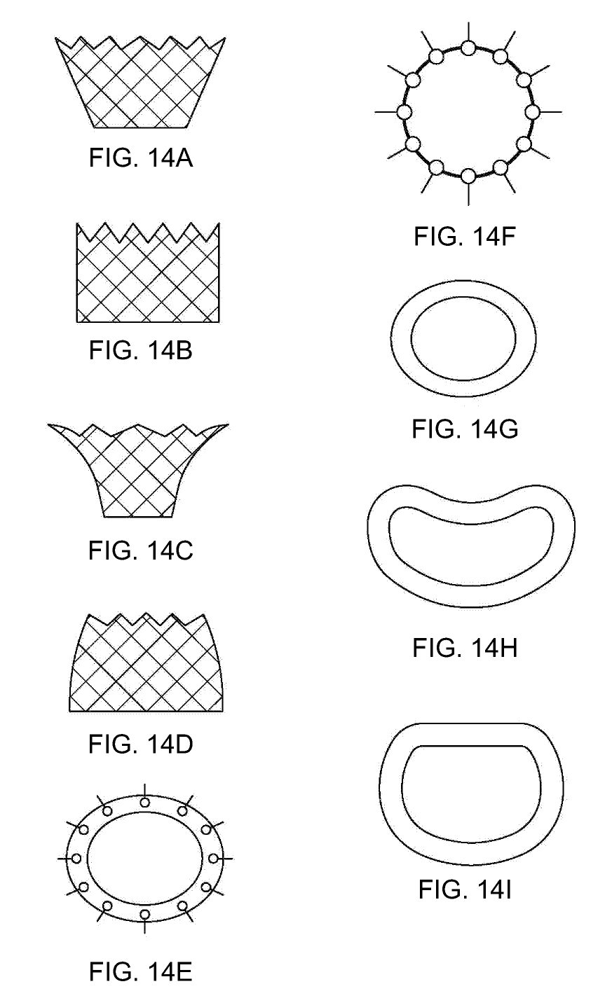

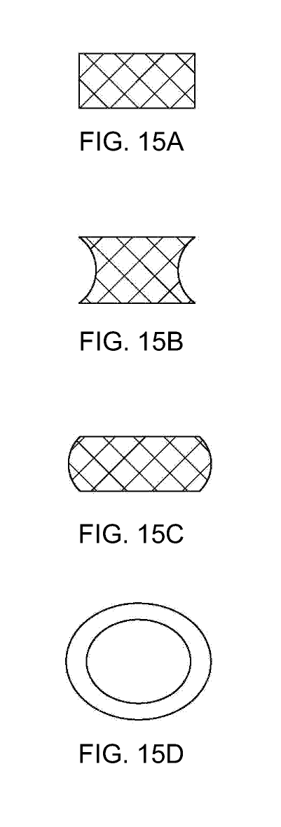

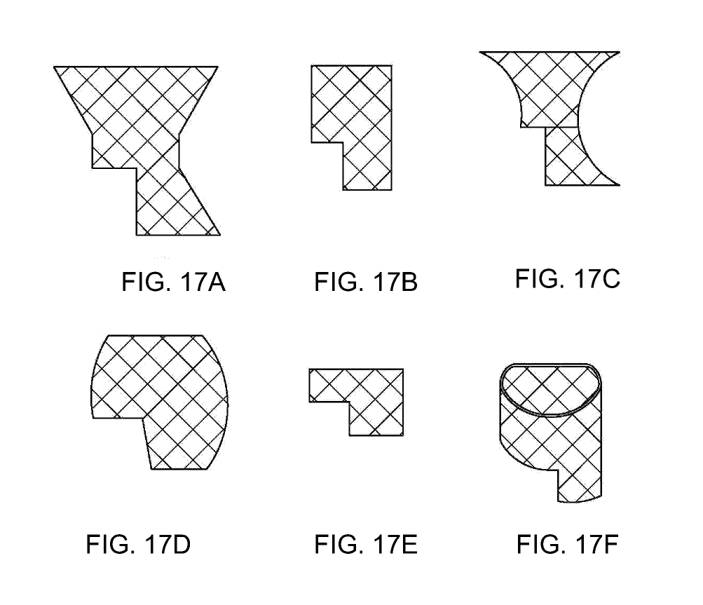

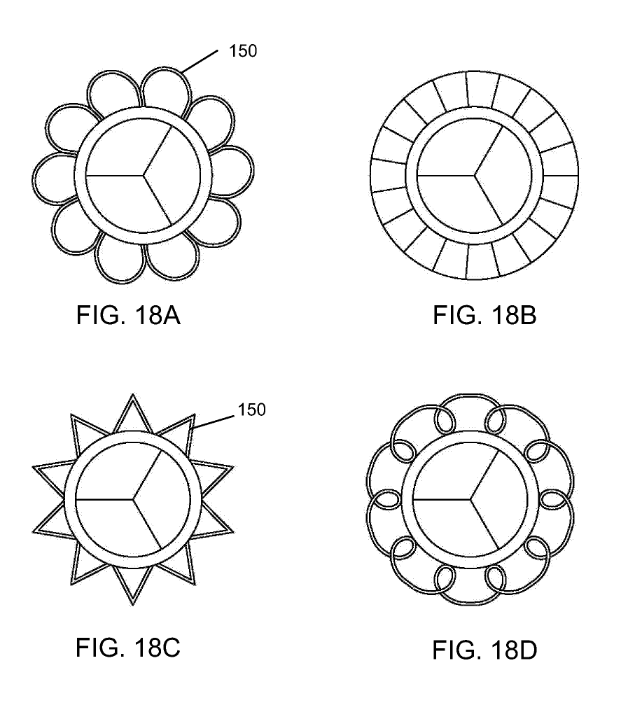

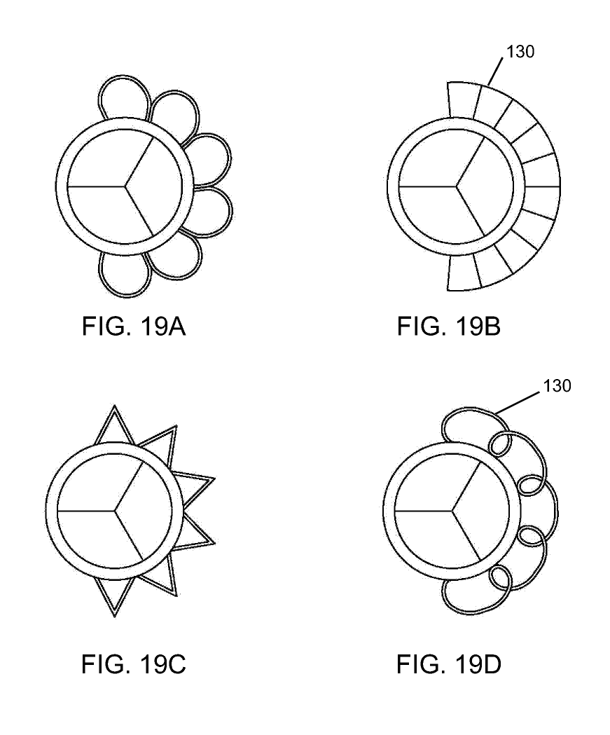

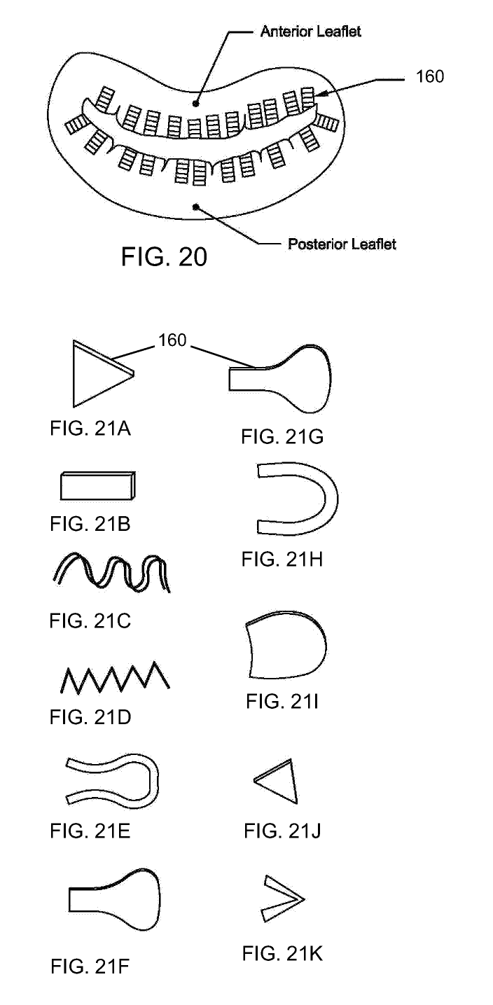

Referring now to FIGS. 1-22, the present invention features a cardiac valve prosthetic (100) effective for use in a living mammalian heart (105) without inducing atrial fibrillation. In one embodiment, the cardiac valve prosthetic (100) comprises a radially, collapsible stent frame (110) and a plurality of valve leaflets (140) internally disposed in the stent frame (110).

In some embodiments, the stent frame (110) comprises a tubular valve portion (120), and a tubular inferior skirt portion (130) fluidly connected to the valve portion (120). The inferior skirt portion (130) may comprise a skirt sidewall (131) and an asymmetrical bottom edge (132). In a preferred embodiment, the valve portion (120) is disposed in a native valve site of the heart (105) and the inferior skirt portion (130) is disposed in a ventricle of the heart.

In one embodiment, the asymmetrical bottom edge (132) is formed by an upper edge section (133) fluidly connected to a lower edge section (134). A first distance (135) is defined as the distance between the upper edge section (133) and a top edge (121) of the valve portion (120). A second distance (136) is defined as the distance between the lower edge section (134) and the top edge (121) of the valve portion (120). Preferably, the first distance (135) is shorter than the second distance (136).

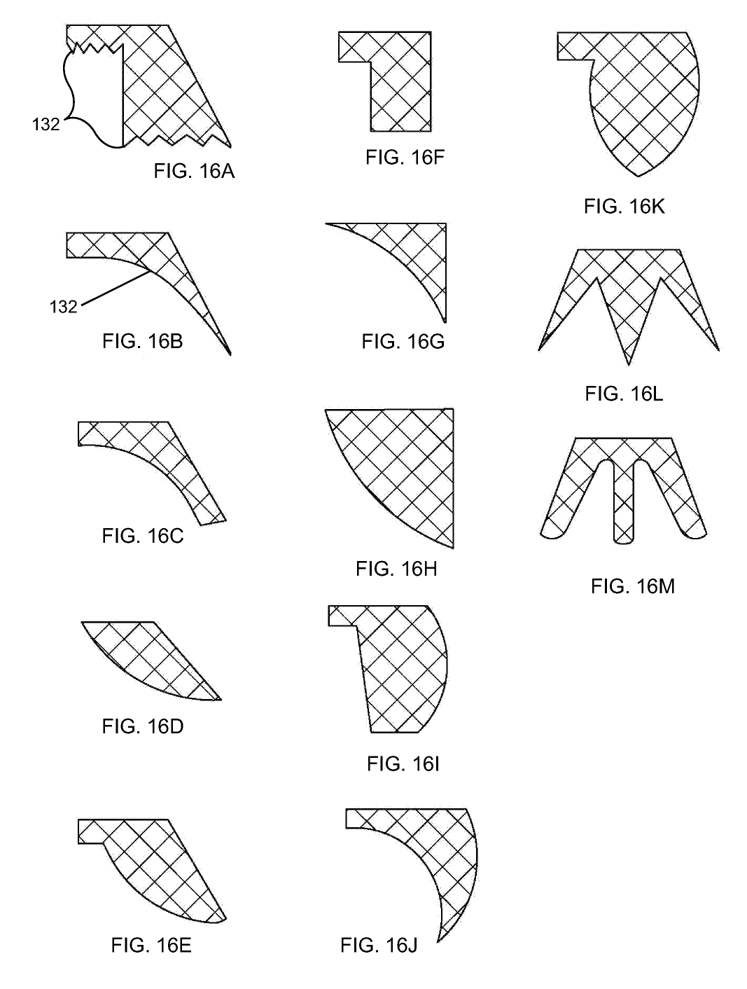

In another embodiment, the asymmetrical bottom edge (132) is formed by at least two upper edge sections (133) interconnected by lower edge sections (134). For example, the bottom edge (132) may comprise alternating upper edge sections (133) and lower edge sections (134) such that there are three upper edge sections (133) and three lower edge sections (134), thereby forming a tripod as shown in FIGS. 16L and 16M. In another example, the bottom edge (132) may comprise alternating upper edge sections (133) and lower edge sections (134) such that there are between about 2-8 upper edge sections (133) and between about 2-8 lower edge sections (134).

In another embodiment, a section of the inferior skirt portion (130) at or near the bottom edge (132) is eliminated from the inferior skirt portion (130) such that the bottom edge (133) is asymmetrical. In still another embodiment, the inferior skirt portion (130) at or near the bottom edge (132) is devoid of structure in the annular (circumferential) direction such that the bottom edge (133) is asymmetrical.

In an alternative embodiment, the inferior skirt portion (130) comprises a first section and a second section that are fluidly connected to form the tubular inferior skirt portion (130). The first section has a first length defined as the longest distance between a first section bottom edge and a first section top edge. The second section has a second length defined as the shortest distance between a second section bottom edge and a second section top edge. The first length is shorter than the second length such that the first section bottom edge and second section bottom edge form the asymmetrical bottom edge (132) of the inferior skirt portion (130).

In some embodiments, the plurality of valve leaflets (140) may be internally disposed in the valve portion (120) of the stent frame (110). In some embodiments, a posterior end (141) of each valve leaflet (140) is attached to the stent frame (110) and a distal end (142) of each valve leaflet (140) is biased towards the inferior skirt portion (130), i.e. the distal end is oriented away from the superior portion of the stent frame. The plurality of valve leaflets (140) may consists of two, three, or four valve leaflets. In some embodiments, the leaflets (140) may be constructed from a sufficiently flexible material. Non-limiting examples of leaflet materials include pericardium or amniotic membranes (porcine, bovine, equine, caprine, ovine, kangaroo, cervid), biological heart valves (porcine, bovine, equine, caprine, ovine, kangaroo, cervid), jugular veins (porcine, bovine, equine, caprine, ovine, kangaroo, cervid), animal-derived materials or manmade materials, polymeric materials, any alloys, ceramic materials, nanoparticles, organic or inorganic materials, or a combination thereof. For example, the leaflets (140) may be made from a combination of materials such as partial animal derived, partial metallic, or partial manmade in any combinations. The leaflets (140) can be individually made and sewn together or fabricated from a tube of tissue. The leaflets (140) may be attached to the stent frame (110) and allow blood to flow in one direction.

In other embodiments, the stent frame (110) may further comprise a tubular superior portion (150) connected to the top edge (121) of the valve portion (120). Preferably, the superior portion is (150) fiuidly connected to the valve portion (120) such that the valve portion (120) is disposed between the superior portion (150) and the inferior skirt portion (130). In one embodiment, the superior portion (150) is disposed in an atrium of the heart for sealing the atrium and receiving blood inflow. In another embodiment, the superior portion (150) may be disposed in a pulmonary artery or in an aorta for blood outflow to exit the prosthetic.

In preferred embodiments, the cardiac valve prosthetic (100) further comprises a plurality of anchors (160) disposed around the stent frame (110). The plurality of anchors (160) is used for mounting the cardiac valve prosthetic (100) to a native valve. In one embodiment, the plurality of anchors (160) may be disposed externally on the stent frame (110). In another embodiment, the plurality of anchors (160) may be disposed around the valve portion (120) of the stent frame (110) to anchor the prosthetic (100) at the native valve site. For example, the anchors (160) may be disposed on at least a portion of an external surface of the valve portion (160) or on at least a portion of the top edge (121) of the valve portion (120). In another embodiment, the plurality of anchors may be disposed around the superior portion (150) of the prosthetic for anchoring the superior portion (150) to at least a portion of a surface of the atrium, aorta, or pulmonary artery. For example, the anchors (160) may be disposed on an external surface or top edge (151) of the superior portion (150). In still another embodiment, the plurality of anchors (160) may be disposed around the inferior skirt portion (130) of the prosthetic, preferably the lower edge portion (134), for anchoring the inferior skirt portion (130) to at least a portion of a surface of the ventricle. However, the plurality of anchors (160) is not limited to the aforementioned configurations and may be configured in any manner suitable for mounting the prosthetic (100).

In some embodiments, the plurality of anchors (160) may comprise hooks or prongs. The plurality of anchors may each, or collectively, have a collapsed configuration for delivery into the heart and an expanded configuration for anchoring to the heart. For example, the anchors (160) may be barbs that are retracted, or curled, when the prosthetic (100) is being delivered to the heart, and then extended, or uncurled, to anchor the prosthetic (100) to the native valve. For example, the anchors (160) may be constructed from metal or polymer materials.

In preferred embodiments, the cardiac valve prosthetic (100) is positioned in a first valve such that the upper edge section (133) of the inferior skirt portion (130) is biased towards a second valve adjacent to the first valve, and the lower edge section (134) is situated away from the second valve, wherein the first valve and second valve are located in a same ventricle. For example, the upper edge section (133) is nearer to the second valve that is adjacent to the first valve, and the lower edge section (134) is oriented away from the second valve such that the lower edge section (134) is further from the second valve. Without wishing to limit the present invention to a particular theory or mechanism, the prosthetic (100) surprisingly does not induce atrial fibrillation.

For example, the cardiac valve prosthetic (100) may be disposed in a mitral valve position such that the upper edge section (133) is biased towards an aortic valve and the lower edge section (134) is positioned away from the aortic valve. In another example, the cardiac valve prosthetic (100) may be disposed in an aortic valve position such that the upper edge section (133) is biased towards a mitral valve and the lower edge section (134) is positioned away from the mitral valve. In another example, the cardiac valve prosthetic (100) may be disposed in a tricuspid valve position such that the upper edge section (133) is biased towards a pulmonary valve and the lower edge section (134) is positioned away from the pulmonary valve. In another example, the cardiac valve prosthetic may be disposed in a pulmonary valve position such that the upper edge section (133) is biased towards a tricuspid valve and the lower edge section (134) is positioned away from the tricuspid valve.

In some embodiments, the cardiac valve prosthetic (100) is generally cylindrical or conical in shape. In other embodiment, the prosthetic (100) is in a shape of an hourglass. For example, the superior portion (150) and inferior skirt portion (130) are generally conical is shape such that the cardiac valve prosthetic (100) tapers at the valve portion. In other embodiments, the cardiac valve prosthetic (100) bulges at the valve portion.

The cardiac valve prosthetic (100) may be constructed from a semi-solid or shape-memory materials. For example, the stent frame (110) can be made from shape memory alloy (SMA), any alloys, stainless steel, any metals (in general), polymeric materials, laboratory created material, ceramic materials, nanoparticles, carbon materials, inorganic materials, organic materials or biologically derived materials such as materials derived from vertebrates and invertebrates. The stent frame (110) can be self-expandable, balloon expandable or expanded with any other aid. The stent frame can be made as one piece, two pieces or from three pieces. For example, if the stent frame (110) is made of three pieces, then the anchors can be present on one or all of the pieces. The stent frame (110) and anchors (160) may be made from same materials or two different materials. The anchors (160) can be made along with the stent frame (110) or separately and attached to the stent frame (110). The cardiac valve (100) prosthetic is not limited to the aforementioned configurations.

In one embodiment, the stent frame (110) is annularly compressible to a first relatively small size that is suitable for catheter (200) delivery of the prosthetic (100) into a patient. For example, the stent frame (110) may be radially compressed such that a diameter of the prosthetic may be between about 3 to 12 mm. In another embodiment, the stent frame (100) is expanded such that the stent frame (100) is pressed against the surface of the native valve, ventricle, or atrium surfaces. The stent frame (110) may be annularly expandable from the first size to a second relatively large size that is suitable for use of the prosthetic (100) in and by a patient. For example, when the stent frame is expanded, the diameter of the prosthetic may be between about 20 to 40 mm.

In some embodiments, the superior portion (150) may have a length of between about 0.25 to 2 cm. For example, the length of the superior portion (150) may be about 1 to 2 cm. In other embodiments, the valve portion (120) may have a length of between about 0.25 to 2 cm. For example, the length of the valve portion (120) may be about 0.25 to 1 cm. In still other embodiments, the inferior skirt portion (130) may have a length of between about 0.25 to 2 cm. For example, the length of the inferior skirt portion (130) may be about 0.5 to 1.5 cm. Preferably, the cardiac valve prosthetic (100) may have a length of between about 0.5 to 6 cm. For example, the cardiac valve prosthetic (100) may have a length of 3 to 6 cm. The cardiac valve prosthetic is not limited to the aforementioned dimensions.

In some embodiments, flexible materials (170), such as polyester fabric or polymeric materials, may be used to cover at least a portion of the stent frame (110) or the entire stent frame (110). In some embodiments, sutures may be used to attach the components together and make the prosthetic functional. For example, sutures may be used to attach the leaflets (140) to the stent frame (110) or to attach a flexible material cover (170) to the stent frame (110). In some embodiments, one type of suture or multiple types of sutures may be used.

Another embodiment of the present invention features a cardiac valve prosthetic (100) effective for use in a living mammalian heart (105) without inducing atriai fibrillation, the cardiac valve prosthetic (100) comprising a radially, collapsible stent frame (110), a plurality of valve leaflets (140) internally disposed in the stent frame (110), and a plurality of anchors (160) disposed around the stent frame (110) for mounting the cardiac valve prosthetic (100) at a native valve site. The plurality of valve leaflets (140) replaces the damaged native valve leaflets. In one embodiment, the radially, collapsible stent frame (110) comprises a tubular superior portion (150) for receiving blood inflow, a tubular inferior skirt portion (1130) comprising a skirt sidewall (131) and an asymmetrical bottom edge (132), and a tubular medial valve portion (120) disposed between the superior portion (150) and the inferior portion (130). The medial valve portion (120) fluidly connects the superior portion (150) and the inferior skirt portion (130) to form the stent frame 9110). The asymmetrical bottom edge (132) of the inferior skirt potion (130) is formed by an upper edge section (133) interconnected or fluidly connected to a lower edge section (134). A first distance (135) between the upper edge section (133) and a top edge (151) of the superior portion (150) is shorter than a second distance (136) between the lower edge section (134) and the top edge (151) of the superior portion (150).

The lower edge section (134) and a portion of the skirt sidewall (131) (i.e. the portion of the skirt sidewall (131) between the lower edge section and the medial valve portion) may form a flap (138) projecting outwardly from the medial valve portion (120). For example, as shown in FIG. 10, the flap (138) may be formed by the lower edge section and a portion of the skirt sidewall that is between the lower edge section and at most half of a bottom edge of the medial valve portion.

A posterior end (141) of each valve leaflet (140) may be attached to the stent frame (110) and a distal end (142) of each valve leaflet (140) may be biased towards the inferior skirt portion (130) and oriented away from the superior portion (150). The embodiment may further comprise a flexible material (170) for covering at least a portion of the stent frame (110).

In a non-limiting example of when the cardiac valve prosthetic (100) is mounted at the native mitral valve site, the superior portion (150) is disposed in a left atrium such that the superior portion (150) seals the left atrium. The medial valve portion (120) is disposed at a native mitral valve, and the inferior skirt portion (130) is disposed in the left ventricle such that the upper edge section (133) is positioned towards an aortic valve and the lower edge section (134) is oriented away from the aortic valve.

In still another embodiment, the present invention features a cardiac valve prosthetic (100) for placement at a mitral valve site of the heart to treat mitral valve insufficiency (regurgitation). This embodiment may comprise any embodiment of the aforementioned stent frame (110). The stent frame (110) is configured to sit in a mitral annulus for sealing the left atrial side and preserving the native structures in the cardiac skeleton (106). This embodiment may also comprise a plurality of anchors (160) to anchor the prosthetic at the mitral annulus. A plurality of leaflets (140) may be attached to the stent frame to function as mitral valve leaflets. This embodiment may further comprise any additional materials necessary to assemble the prosthetic, such as polyester fabric, tabs, or sutures.

The present invention can also feature a method of implanting a cardiac valve prosthetic (100) in a living mammalian heart (105). In one embodiment, the method may comprise the steps of providing the cardiac valve prosthetic (100), delivering the cardiac valve prosthetic (100) to a native mitral valve site, positioning the cardiac valve prosthetic (100) at the native mitral valve site such that the medial valve portion (120) is positioned at a native mitral valve annulus, radially expanding the superior portion (150) in a left atrium such that the superior portion (150) is anchored to at least a portion of the left atrium and the superior portion (150) seals the left atrium, anchoring the medial valve portion (120) to the native mitral valve annulus via the plurality of anchors, radially expanding the medial valve portion (120) such that the native mitral valve leaflets are displaced, and radially expanding the inferior skirt portion (130) in a left ventricle such that the inferior skirt portion (130) is anchored to at least a portion of the left ventricle. In a preferred embodiment of the method, the upper edge section (133) is positioned towards an aortic valve and the flap (138) is oriented away from the aortic valve such that the aortic valve is unobstructed. Moreover, the method of implanting the cardiac valve prosthetic (100) may eliminate atrial fibrillation.

In another embodiment of the present invention, a method of implanting a cardiac valve prosthetic (100) in a living mammalian heart (105) may comprise the steps of positioning and anchoring the cardiac valve prosthetic (100) at a mitral valve site. The superior portion (150) of the prosthetic is disposed in a left atrium such that the superior portion (150) seals the left atrium. The medial valve portion (120) is disposed at a native mitral valve annulus, and may be anchored to the native mitral valve annulus via anchors (180). Preferably, the inferior skirt portion (130) is disposed in the left ventricle such that the upper edge section (133) is positioned towards an aortic valve and the flap (138) is oriented away from the aortic valve.

The methods for implanting the cardiac valve prosthetic (100) may comprise any embodiments of the cardiac valve prosthetic (100) as described herein. For example, the cardiac valve prosthetic (100) may comprise a radially, collapsible stent frame (110), a plurality of valve leaflets (140), and a plurality of anchors (160) disposed around the stent frame (110). In some embodiments, the radially, collapsible stent frame (110) may comprise a tubular superior portion (150) for receiving blood inflow, a tubular inferior skirt portion (130) comprising a skirt sidewall (131) and an asymmetrical bottom edge (132), and a tubular medial valve portion (120) disposed between the superior portion (150) and the inferior portion (130). The medial valve portion (120) can fluidly connect the superior portion (150) and the inferior skirt portion (130) to form the stent frame (110). In other embodiments, the bottom edge (132) may be formed by an upper edge section (133) fluidly connected to a lower edge section (134). A first distance (135) between the upper edge section (133) and a top edge (151) of the superior portion (150) is shorter than a second distance (136) between the lower edge section (134) and the top edge (151) of the superior portion (150). In still other embodiments, the lower edge section (134) and a portion of the skirt sidewall (131) forms a flap (138) projecting outwardly from a tubular medial valve portion (120). In some embodiments, the plurality of valve leaflets (140) may be internally disposed in the medial valve portion (120) of the stent frame (110) such that a posterior end (141) of each valve leaflet (140) is attached to the stent frame (110) and a distal end (142) of each valve leaflet (140) is biased towards the inferior skirt portion (130).

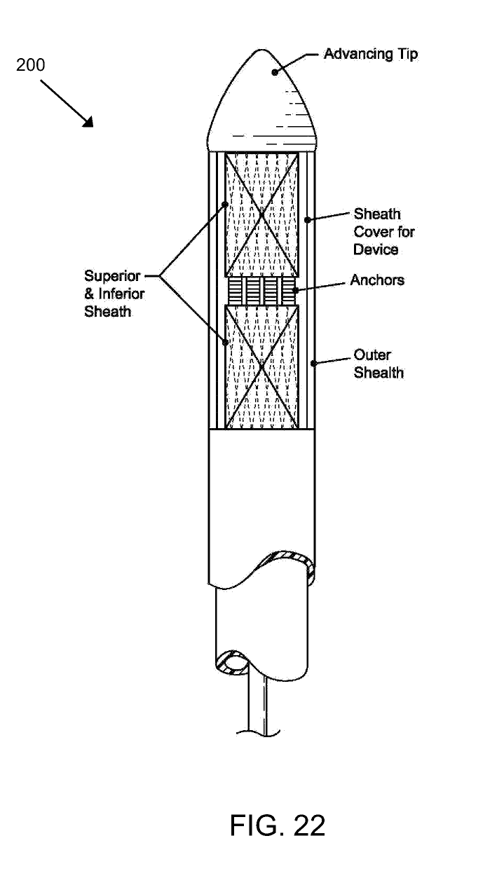

Moreover, the methods for implanting the cardiac valve prosthetic (100) may comprise any suitable delivery system for delivering the cardiac valve prosthetic. A non-limiting example of such delivery system is shown in FIG. 22 and may comprise a catheter (200).

In some embodiments, the cardiac valve prosthetic (100) of the present invention can perform the following functions when deployed.

1. The superior portion (150) of the prosthetic can expand and seal the atrial side of the mitral valve and prevent any leakage.

2. The medial valve portion (120) of the prosthetic can anchor to the mitral annulus. The medial valve portion may contain leaflets to fake over the function of the diseased native leaflets. These replacement leaflets may treat the mitral valve insufficiency (regurgitation) by only allowing a one-way flow of blood from the left atrium to the left ventricle.

3. The inferior skirt portion (130) of the prosthetic can expand, support, and keep the prosthetic in position. The inferior skirt portion does not expand around the entire mitral annulus because a part of the inferior skirt portion is omitted. The remaining part of the inferior skirt portion expands and is located away from the aorta.

As used herein, the term “about” refers to plus or minus 10% of the referenced number.

The disclosures of U.S. Patents and Publications disclosed herein are incorporated in their entirety by reference. Each reference cited in the present application is incorporated herein by reference in its entirety.

Various modifications of the invention, in addition to those described herein, will be apparent to those skilled in the art from the foregoing description. Such modifications are also intended to fall within the scope of the appended claims.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

### REFERENCES

1. Amat-Santos, I. et al. Incidence, Predictive Factors, and Prognostic Value of New-onset Atrial Fibrillation Following Transcatheter Aortic Valve Implantation. J Am Coll Cardiol. 2012. 59(2); 178-188.

What is claimed is:

1. A cardiac valve prosthetic (100) effective for use in a living mammalian heart (105) without inducing atrial fibrillation, the cardiac valve prosthetic (100) comprising:
a. a radially, collapsible stent frame (110) comprising:
i. a tubular valve portion (120); and
ii. a tubular inferior skirt portion (130) fluidly connected to the valve portion (120), wherein the inferior skirt portion (130) comprises a skirt sidewall (131) and an asymmetrical bottom edge (132), wherein the bottom edge (132) is formed by an upper edge section (133) fluidly connected to a lower edge section (134), wherein a first distance (135) between the upper edge section (133) and a top edge (121) of the valve portion (120) is shorter than a second distance (136) between the lower edge section (135) and the top edge (121) of the valve portion (120); and
b. a plurality of valve leaflets (140) internally disposed in the valve portion (120) of the stent frame (110), wherein a posterior end (141) of each valve leaflet (140) is attached to the stent frame (110) and a distal end (142) of each valve leaflet (140) is biased towards the inferior skirt portion (130).

2. The cardiac valve prosthetic (100) of claim 1, wherein the stent frame further comprises a tubular superior portion (150) for receiving blood inflow, wherein the superior portion (150) is fluidly connected to the valve portion (120) such that the valve portion (120) is disposed between the superior portion (150) and the inferior skirt portion (130).

3. The cardiac valve prosthetic (100) of claim 2, wherein the superior portion (150) is disposed in an atrium, wherein the superior portion (150) seals the atrium.

4. The cardiac valve prosthetic (100) of claim 1 further comprising a plurality of anchors (160) attached to the stent frame (110) for mounting the cardiac valve prosthetic (100) to a native valve.

5. The cardiac valve prosthetic (100) of claim 1, wherein the valve portion (120) is disposed in a native valve site of the heart (105).

6. The cardiac valve prosthetic (100) of claim 1, wherein the inferior skirt portion (130) is disposed in a ventricle of the heart.

7. The cardiac valve prosthetic (100) of claim 1, wherein the cardiac valve prosthetic (100) is positioned in a first valve such that the upper edge section (133) of the inferior skirt portion (130) is biased towards a second valve adjacent to the first valve, and the lower edge section (134) is situated away from the second valve, wherein the first valve and second valve are located at a same ventricle.

8. The cardiac valve prosthetic (100) of claim 1, wherein the cardiac valve prosthetic (100) is disposed in a mitral valve position such that the upper edge section (133) is biased towards an aortic valve and the lower edge section (134) is positioned away from the aortic valve.

9. The cardiac valve prosthetic (100) of claim 1, wherein the cardiac valve prosthetic (100) is disposed in an aortic valve position such that the upper edge section (133) is biased towards a mitral valve and the lower edge section (134) is positioned away from the mitral valve.

10. The cardiac valve prosthetic (100) of claim 1, wherein the cardiac valve prosthetic (100) is disposed in a tricuspid valve position such that the upper edge section (133) is biased towards a pulmonary valve and the lower edge section (134) is positioned away from the pulmonary valve.

11. The cardiac valve prosthetic (100) of claim 1, wherein the cardiac valve prosthetic (100) is disposed in a pulmonary valve position such that the upper edge section (133) is biased towards a tricuspid valve and the lower edge section (134) is positioned away from the tricuspid valve.

12. The cardiac valve prosthetic (100) of claim 2, wherein the superior portion (150) and inferior skirt portion (130) are generally conical is shape such that the cardiac valve prosthetic (100) tapers at the valve portion (120).

13. The cardiac valve prosthetic (100) of claim 1, wherein the cardiac valve prosthetic (100) bulges at the valve portion (120).

14. The cardiac valve prosthetic (100) of claim 1, wherein the cardiac valve prosthetic (100) is made from a semi-solid or shape-memory material.

15. The cardiac valve prosthetic (100) of claim 1 further comprising a flexible material (170) attached to and covering at least a portion of the stent frame (110).

16. The cardiac valve prosthetic (100) of claim 1, wherein the stent frame (110) is annularly compressible to a first relatively small size, wherein the first size is suitable for delivery of the prosthetic (100) into a patient via a catheter (200).

17. The cardiac valve prosthetic (100) of claim 1, wherein the stent frame (110) is annularly expandable from the first size to a second relatively large size, wherein the second size is suitable for use of the prosthetic (100) in and by a patient.

18. A cardiac valve prosthetic (100) effective for use in a living mammalian heart (105) without inducing atrial fibrillation, the cardiac valve prosthetic (100) comprising:
a. a radially, collapsible stent frame (110) comprising:
i. a tubular superior portion (150) for receiving blood inflow;
ii. a tubular inferior skirt portion (130) comprising a skirt sidewall (131) and an asymmetrical bottom edge (132), wherein the bottom edge (132) is formed by an upper edge section (133) fluidly connected to a lower edge section (134), wherein a first distance (135) between the upper edge section (133) and a top edge (151) of the superior portion (150) is shorter than a second distance (136) between the lower edge section (134) and the top edge (151) of the superior portion (150); and
iii. a tubular medial valve portion (120) disposed between the superior portion (150) and the inferior portion (130), wherein the medial valve portion (120) fluidly connects the superior portion (150) and the inferior skirt portion (130) to form the stent frame (110); and
b. a plurality of valve leaflets (140) internally disposed in the medial valve portion (120) of the stent frame (110), wherein a posterior end (141) of each valve leaflet (140) is attached to the stent frame (110) and a distal end (142) of each valve leaflet (140) is biased towards the inferior skirt portion (130), wherein the plurality of valve leaflets (140) replaces the native valve leaflets;
c. a plurality of anchors (160) disposed around the stent frame (110) for mounting the cardiac valve prosthetic (100) at a native valve site; and
d. a flexible material (170) for covering at least a portion of the stent frame (110);
wherein the lower edge section (134) and a portion of the skirt sidewall (131) forms a flap (138) projecting outwardly from the medial valve portion (120); and
wherein when the cardiac valve prosthetic (100) is mounted at the native mitral valve site, the superior portion (150) is disposed in a left atrium such that the superior portion (150) seals the left atrium, wherein the medial valve portion (120) is disposed at a native mitral valve, and wherein the inferior skirt portion (130) is disposed in the left ventricle such that the upper edge section (133) is positioned towards an aortic valve and the lower edge section (134) is oriented away from the aortic valve.

19. A method of implanting a cardiac valve prosthetic (100) in a living mammalian heart (105), said method comprising:
a. providing the cardiac valve prosthetic (100), wherein the cardiac valve prosthetic (100) comprises:
i. a radially, collapsible stent frame (110) comprising:
1) a tubular superior portion (150) for receiving blood inflow;
2) a tubular inferior skirt portion (130) comprising a skirt sidewall (131) and an asymmetrical bottom edge (132), wherein the bottom edge (132) is formed by an upper edge section (133) interconnected to a lower edge section (134), wherein a first distance (135) between the upper edge section (133) and a top edge (151) of the superior portion (150) is shorter than a second distance (136) between the lower edge section (134) and the top edge (151) of the superior portion (150), wherein the lower edge section (134) and a portion of the skirt sidewall (131) forms a flap (138) projecting outwardly from a tubular medial valve portion (120); and
3) the tubular medial valve portion (120) disposed between the superior portion (150) and the inferior portion (130), wherein the medial valve portion (120) fiuidly connects the superior portion (150) and the inferior skirt portion (130) to form the stent frame (110); and
ii. a plurality of valve leaflets (140) internally disposed in the medial valve portion (120) of the stent frame (110), wherein a posterior end (141) of each valve leaf let (140) is attached to the stent frame (110) and a distal end (142) of each valve leaflet (140) is biased towards the inferior skirt portion (130); and
iii. a plurality of anchors (160) disposed around the stent frame (110);
b. delivering the cardiac valve prosthetic (100) to a native mitral valve site via a transcatheter delivery system;
c. positioning the cardiac valve prosthetic (100) at the native mitral valve site such that the medial valve portion (120) is positioned at a native mitral valve annulus;
d. radially expanding the superior portion (150) in a left atrium such that the superior portion (150) is anchored to at least a portion of the left atrium and the superior portion (150) seals the left atrium;
e. anchoring the medial valve portion (120) to the native mitral valve annulus via the plurality of anchors;
f. radially expanding the medial valve portion (120) such that the native mitral valve leaflets are displaced, wherein the plurality of valve leaflets (140) replaces the native mitral valve leaflets; and
g. radially expanding the inferior skirt portion (130) in a left ventricle such that the inferior skirt portion (130) is anchored to at least a portion of the left ventricle, wherein the upper edge section (133) is positioned towards an aortic valve and the flap (138) is oriented away from the aortic valve such that the aortic valve is unobstructed;
wherein the method of implanting the cardiac valve prosthetic (100) eliminates atrial fibrillation.

20. A method of implanting a cardiac valve prosthetic (100) in a living mammalian heart (105), said method comprising positioning and anchoring the cardiac valve prosthetic (100) at a mitral valve site, wherein the cardiac valve prosthetic comprises:
a. a radially, collapsible stent frame (110) comprising:
i. a tubular superior portion (150) for receiving blood inflow;
ii. a tubular inferior skirt portion (130) comprising a skirt sidewall (131) and an asymmetrical bottom edge (132), wherein the bottom edge (132) is formed by an upper edge section (133) interconnected to a lower edge section (134), wherein a first distance (135) between the upper edge section (133) and a top edge (151) of the superior portion (150) is shorter than a second distance (136) between the lower edge section (134) and the top edge (151) of the superior portion (150), wherein the lower edge section (134) and a portion of the skirt sidewall (131) form a flap (138) projecting outwardly from a tubular medial valve portion (120); and
iii. the tubular medial valve portion (120) disposed between the superior portion (150) and the inferior portion (130), wherein the medial valve portion (120) fluidly connects the superior portion (150) and the inferior skirt portion (130) to form the stent frame (110); and
b. a plurality of valve leaflets (140) internally disposed in the medial valve portion (120) of the stent frame (110), wherein a posterior end (141) of each valve leaflet (140) is attached to the stent frame (110) and a distal end (142) of each valve leaflet (140) is biased towards the inferior skirt portion (130); and
c. a plurality of anchors (160) disposed around the stent frame (110);
wherein the superior portion (150) is disposed in a left atrium such that the superior portion (150) seals the left atrium, wherein the medial valve portion (120) is disposed at a native mitral valve annulus, and wherein the inferior skirt portion (130) is disposed in the left ventricle such that the upper edge section (133) is positioned towards an aortic valve and the flap (138) is oriented away from the aortic valve.
